(19) United States
(12) Patent Application Publication
(10) Pub. No.: US 2024/0265557 A1
(43) Pub. Date: Aug. 8, 2024

(54) MEDICAL INFORMATION PROCESSING DEVICE, MEDICAL INFORMATION PROCESSING METHOD AND NON-TRANSITORY COMPUTER READABLE MEDIUM

(71) Applicant: CANON MEDICAL SYSTEMS CORPORATION, Otawara-shi (JP)

(72) Inventors: Shinya NISHITAKA, Nasushiobara (JP); Takahiko NISHIOKA, Otawara (JP)

(73) Assignee: CANON MEDICAL SYSTEMS CORPORATION, Otawara-shi (JP)

(21) Appl. No.: 18/435,043

(22) Filed: Feb. 7, 2024

(30) Foreign Application Priority Data
Feb. 8, 2023 (JP) 2023-017785
Feb. 1, 2024 (JP) 2024-014438

(51) Int. Cl. G06T 7/33; A61B 6/51; A61C 13/34; G06T 7/55; G06T 15/08; G06T 19/20

(52) U.S. Cl. CPC G06T 7/33; A61B 6/512; A61C 13/34; G06T 7/55; G06T 15/08; G06T 19/20; G06T 2207/10024; G06T 2207/10081; G06T 2207/20212; G06T 2207/30036; G06T 2210/41; G06T 2210/62; G06T 2219/2012

(57) ABSTRACT

A medical information processing device includes a memory and a processor. The processor is configured to generate a volume rendering (VR) image from a medical image of a subject; acquire a color image acquired by one or a plurality of optical imaging units, the color image sharing at least a part of an imaging region with the medical image; and superimpose at least a part of the color image on the VR image.

### CROSS REFERENCE TO THE RELATED APPLICATIONS

This application is based upon and claims the benefit of priority from the Japanese Patent Application No. 2023-017785, filed on Feb. 8, 2023, and the Japanese Patent Application No. 2024-014438, filed on Feb. 1, 2024, the entire contents of which are incorporated herein by reference.

### FIELD

Embodiments disclosed in the present specification and the drawings relate to a medical information processing device, a medical information processing method, and a non-transitory computer readable medium.

### BACKGROUND

In dentistry diagnosis, an image photographed by a digital camera and an image photographed by an X-ray are often used. In addition thereto, an image photographed using an X-ray computed tomography (CT) device is increasingly used today. By displaying an image of an X-ray CT by volume rendering (VR), it is possible to observe internal information in an oral cavity from various directions. In addition, since it is possible to check, by the X-ray CT, the internal information of teeth and gums that cannot be visually checked, the X-ray CT is indispensable for diagnosis of dental caries and diagnosis of periodontal disease today.

However, since it is difficult to check, from the X-ray CT image, the state of the surface of the teeth or the gums that can be visually checked, there is a problem in that it is difficult to grasp the actual state of the patient's teeth. In addition, simulation of a prosthesis or an implant after treatment is also advantageous in patient description when VR display is used, but there is also a problem in that it is difficult to make an image because a color of the VR display is different from an actual teeth color.

### BRIEF DESCRIPTION OF DRAWINGS

FIG. 1 is a block diagram schematically illustrating an example of a medical information processing device according to an embodiment.

FIG. 2 is a diagram illustrating an example of an image of a VR model according to the embodiment as viewed from a predetermined angle.

FIG. 3 is a diagram illustrating an example of a color image according to the embodiment.

FIG. 4 is a diagram illustrating an example of a composite image according to the embodiment.

FIG. 5 is a flowchart illustrating processing of the medical information processing device according to the embodiment.

FIG. 6 is a diagram illustrating an example of a color image according to the embodiment.

FIG. 7 is a flowchart illustrating processing of an alignment function according to the embodiment.

FIG. 8 is a diagram illustrating an example of a position of alignment according to the embodiment.

FIG. 9 is a flowchart illustrating processing of an overlay function according to the embodiment.

FIG. 10 is a diagram illustrating an example of a composite image according to the embodiment.

FIG. 11 is a diagram schematically illustrating an example of a GUI of the medical information processing device according to the embodiment.

### DETAILED DESCRIPTION

According to one embodiment, a medical information processing device includes a memory and a processor. The processor is configured to generate a volume rendering (VR) image from a medical image of a subject; acquire a color image acquired by one or a plurality of optical imaging units, the color image sharing at least a part of an imaging region with the medical image; and superimpose at least a part of the color image on the VR image.

Hereinafter, embodiments will be described with reference to the drawings. In the present disclosure, in a mode in which information processing by software is specifically implemented by using hardware resources, the software is executed by a program, and can be implemented by the program itself or a non-transitory computer readable medium storing the program. Furthermore, in the various data in the present specification and the drawings, data mainly used by an information processing device is typically digital data.

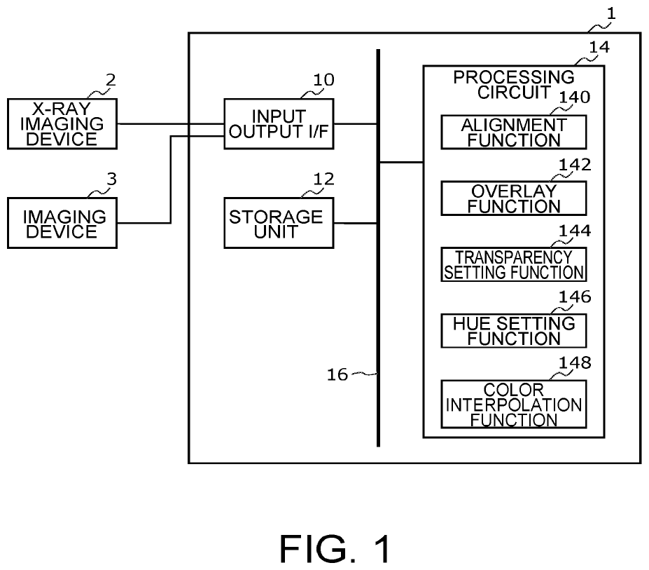

FIG. 1 is a block diagram schematically illustrating an example of a medical information processing device according to an embodiment. A medical information processing device 1 is a device directly or indirectly connected to an X-ray imaging device 2 that photographs an X-ray image and an imaging device 3 that photographs an image in a visible-light band, and generating an image obtained by combining (fusing) and superimposing (overlaying) color images on at least a part of a VR CT image generated from the X-ray image. The medical information processing device 1 includes an input/output I/F 10, a storage unit 12, and a processing circuit 14.

The input/output I/F 10 is an interface for connecting the inside and the outside of the medical information processing device 1. The input/output I/F 10 includes, for example, a communication interface for receiving an image based on X-rays output from the X-ray imaging device 2 and a color image output from the imaging device 3.

In addition, the input/output I/F 10 can include input interfaces such as a keyboard, a mouse, a trackball, a touch pad, a button, a microphone, and a camera that receive an input from a user, or an interface for being connected to these input interfaces, and output interfaces such as a display and a stereo that output information to a user, or an interface for being connected to these output interfaces.

The storage unit 12 stores data acquired from the outside via the input/output I/F 10, data required for processing in the processing circuit 14, temporary intermediate data in the processing of the processing circuit 14, processed data, and data for the processing circuit 14 to perform each function. Although not illustrated, a storage device may also be provided outside the medical information processing device 1, and various data may be stored in the storage device.

The processing circuit 14 is a circuit that executes each processing in the medical information processing device 1. The processing circuit 14 maybe, for example, a dedicated circuit or a general-purpose processor. The processing circuit 14 implements, for example, an alignment function 140, an overlay function 142, a transparency setting function 144, a hue setting function 146, and a color interpolation function 148. It is noted that functions other than the alignment function 140 and the overlay function 142 are functions that can be freely and selectively implemented, and are not essential to the processing circuit 14.

The alignment function 140 is a function of performing alignment of superimposing a color image on a VR image acquired from X-ray CT. For example, the alignment function 140 extracts features common to the VR image and the color image, and performs alignment so as to superimpose points, lines, or regions having these features. The medical information processing device 1 can include the alignment function 140 as an alignment unit.

The overlay function 142 is a function of performing processing of superimposing the color image on at least a partial region on the VR image. The overlay function 142 superimposes an image region to be superimposed extracted from the color image on the VR image based on alignment information acquired by the alignment function 140. The medical information processing device 1 can include the overlay function 142 as an overlay unit.

The transparency setting function 144 is a function of setting transparency of a color image region to be superimposed. The transparency setting function 144 freely and selectively sets transparency of a color image to be superimposed, for example, in response to a request from a user, and enables generation of an image that can be viewed through the VR image to be superimposed. The medical information processing device 1 can include the transparency setting function 144 as a transparency setting unit.

The hue setting function 146 is a function of setting a hue of a color image region to be superimposed. The hue setting function 146 can freely and selectively set a hue of a color image to be superimposed, for example, according to a color extracted from the color image or in response to a request from a user. Further, the hue setting function 146 may have a function of setting a hue of a predetermined region in the color image to be superimposed. The medical information processing device 1 can include the hue setting function 146 as a hue setting unit.

The color interpolation function 148 is a function of estimating, from surrounding color information, color information on a region in which color information cannot be appropriately acquired in the color image region to be superimposed, and interpolating the color information thereon. The color interpolation function 148 can interpolate a color based on various image interpolation units. The medical information processing device 1 can include the color interpolation function 148 as a color interpolation unit.

The processing circuit 14 superimposes at least a partial region of the color image on the VR image generated from the X-ray CT by using at least the alignment function 140 and the overlay function 142 among the above brain functions. The processing circuit 14 can freely and selectively set the transparency of the color image, set the hue, or interpolate the colors to perform superimposition.

The X-ray imaging device 2 acquires an X-ray CT image of any region of the patient's body. The X-ray imaging device 2 may further generate a VR model from the acquired CT image. As another example, generation of VR from the CT image may be implemented by the processing circuit 14 of the medical information processing device 1 or may be executed by another information processing device (not illustrated). In the following description, the X-ray imaging device 2 generates VR, but similarly, the generation of the VR by the medical information processing device 1 or another information processing device is not excluded. Hereinafter, an image obtained by acquiring the VR model from any angle is referred to as a VR image.

The medical information processing device 1 can implement superimposition of images in various forms capable of acquiring an X-ray CT image and a color image acquired by an optical imaging unit. Hereinafter, particularly, an image in the oral cavity in dentistry will be described, but the embodiment of the present disclosure is not limited to dentistry, and application to other medical images is not excluded. Hereinafter, when a description is simply given as a color image in the present specification, the color image can be read as a color image acquired by an optical imaging unit as in this paragraph. Further, the same applies to the drawings.

In dentistry diagnosis, a three-dimensional VR model may be formed from an X-ray CT image in an oral cavity of a patient. The medical information processing device 1 generates, at a timing of acquiring an image of any angle of this formed VR model, a composite image by superimposing a color image on at least a part thereof.

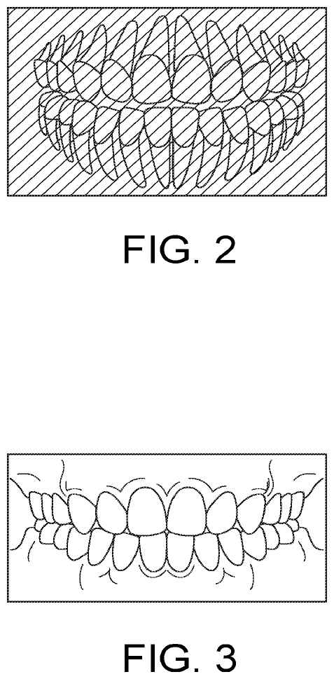

FIG. 2 is a diagram illustrating an example of an image of a VR model viewed from a predetermined angle (for example, the front side of the face). In the present disclosure, in order to clearly show an image according to the X-ray and a color image, a region of a VR image is indicated by hatching.

As illustrated in FIG. 2, the image acquired from the VR model represents skeleton information indicating teeth, teeth roots, and the like. The X-ray imaging device 2 reconstructs a three-dimensional model from the acquired X-ray CT images of various angles, and generates the VR model. The overlay function 142 of the processing circuit 14 acquires an image of an angle selected by a user from the input VR model. For example, in a case where the front is selected, the medical information processing device 1 can acquire an image as illustrated in FIG. 2.

FIG. 3 is a diagram illustrating an example of a color image. Based on alignment information acquired by the alignment function 140, the overlay function 142 cuts off at least a partial region of the color image illustrated in FIG. 3 and superimposes the cut-off partial region on the VR image, so that the processing circuit 14 can generate a composite image simultaneously indicating a region in which the color image is desired to be viewed and a region in which the VR image is desired to be viewed.

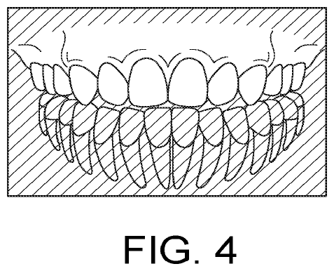

FIG. 4 is a diagram illustrating an example of a composite image according to the embodiment. As an example, as illustrated in FIG. 4, the medical information processing device 1 can generate an image in which a portion of the upper teeth in the oral cavity is displayed as a color image and the other part in the oral cavity is displayed as a VR image.

By referring to such a composite image, it is possible to check internal information of the teeth or gums with reference to the VR image, to acquire information such as dental caries and implants and bridges of already treated teeth, and to display information such as prostheses and implants after treatment as a color image.

Further, in the diagnosis as well, by using the composite image, information on the surfaces of the teeth can be viewed as the color image together with the internal information that can be viewed by the X-ray, so that weak points in the images can be complementarily reinforced.

Hereinafter, each function implemented in the processing circuit 14 will be described in detail while referring to an overall processing flow.

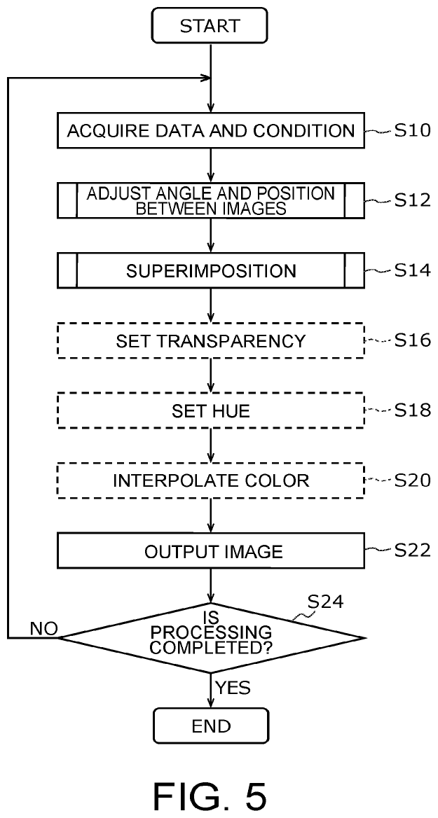

FIG. 5 is a flowchart illustrating processing of the medical information processing device 1 according to the embodiment.

The medical information processing device 1 acquires, in response to a request from a user, VR model data of a VR image and image data of color image data, and a request for an image generation condition such as an angle at which an image is generated (S10). If necessary, the condition may include setting of transparency and hue. By this processing, a VR image and a color image to be superimposed can be acquired, and a condition regarding an image to be generated can be acquired.

The medical information processing device 1 may acquire an image photographed by, for example, a so-called five-image method for data of the color image.

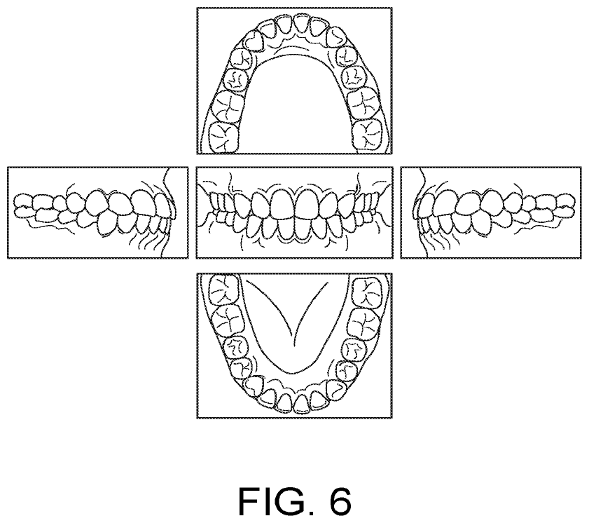

FIG. 6 is a diagram illustrating an example of the color image. As illustrated in this drawing, the color image in the oral cavity is often photographed by the five-image method. A predetermined quality can be maintained by performing photographing by the five-image method according to the standard. In a case where an image is photographed with a predetermined quality, it is also possible to simplify alignment processing described below, and it is also possible to improve accuracy.

For example, in the front image shown at the center of the images, an image is taken such that the midline of the upper teeth is located vertically at the center of the image and the underline of the upper front teeth is located horizontally at the center of the image. In a case where such photographing is performed, it is possible to improve accuracy of vertical and horizontal angle alignment and alignment in a case where the front image is used. In the following description, a description will be given as to an example in which an appropriate angle and position can be adjusted even in a case of not conforming to such a standard.

Referring back to FIG. 5, the alignment function 140 adjusts the angle and the position between the VR image and the color image (S12).

### Alignment Function

For example, the alignment function 140 adjusts the angle and the position of an image when the VR image illustrated in FIG. 2 and the color image illustrated in FIG. 3 are superimposed. The alignment function 140 can use teeth as a feature used for alignment in the VR image and the color image.

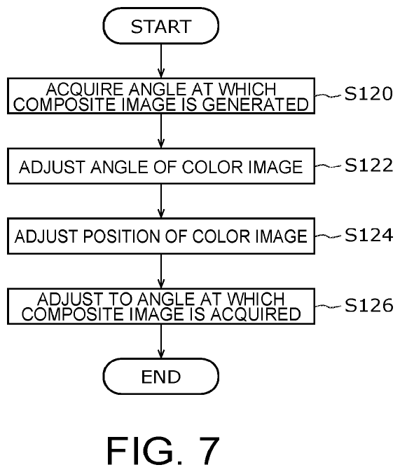

FIG. 7 is a flowchart illustrating processing of the alignment function according to the embodiment. Although an example of adjusting and converting the color image in accordance with the VR image will be described, the VR image may be adjusted and converted in accordance with the color image, or any image to be adjusted and converted may be changed by depending on processing.

The alignment function 140 acquires information on an angle at which a composite image requested by a user is generated (S120). The angle at which the composite image is generated is information indicating from what viewpoint the composite image is generated.

The alignment function 140 adjusts the angle of the color image to be equal to the angle of the VR image (S122). For example, in the front image, the alignment function 140 extracts two end points of the upper front teeth (the lower front teeth may be used if a patient is an opposite bite) by any feature point extraction unit, and rotates the color image around the axial direction perpendicular to the image such that an inclination of a straight line connecting the extracted two end points is equal to that of the VR image acquired from the front.

Subsequently, for example, the alignment function 140 calculates areas of the two upper front teeth by any method for each of the VR image and the color image, and acquires a ratio of the areas of the two front teeth. The image is rotated around the midline direction (for example, the vertical direction in the image) such that the ratios are equal. In a case where an image is acquired by the five-image method, this rotation can be executed by combining an image photographed from one of the right and left directions and an image photographed from the front direction by any method, for example, any method using projection transformation.

The alignment function 140 shifts the color image in the vertical direction and the horizontal direction such that corners of the tips of the front teeth, for example, the inner tips of the two front teeth overlap each other in the VR image and the color image (S124).

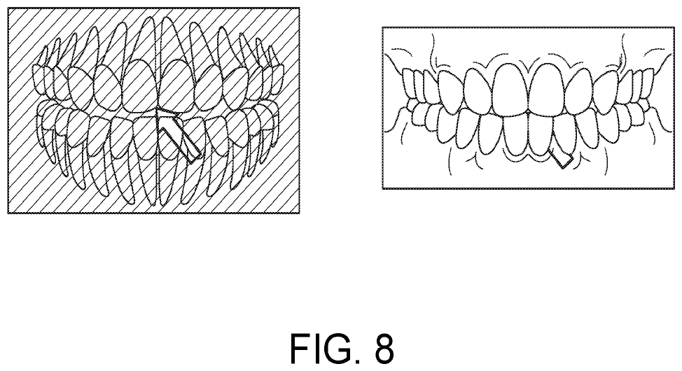

FIG. 8 is a diagram illustrating an example of a position serving as a reference of this alignment. For example, as indicated by arrows in FIG. 8, the positions of the color images can be adjusted such that the end points of the front teeth in the respective images overlap each other.

The alignment function 140 can adjust the VR image and the color image to be images acquired from the same direction and from the same position by the processing in S122 and S124.

It is noted that, in a case where the sizes of the VR image and the color image are different from each other, the alignment function 140 can also enlarge and reduce, based on the area of the front teeth of each of the VR image and the color image, four end points in a case where the image is regarded as a rectangle, and the like, the size of the color image at a magnification corresponding to the size of the VR image at this timing.

Depending on the angle at which the composite image is generated, the color image to be used may be changed from the front image to an image photographed from one of the right and left angles. In this case, a reference position is determined in advance, and the angle and the position can be adjusted based on the reference position in the same manner as described above. For example, the alignment function 140 can perform the above-described angle alignment based on the contour, the area, and the like of a tooth adjacent to a tooth including the reference position or the two teeth close to the reference position.

As some non-limiting examples, the alignment function 140 can also perform alignment as shown below.

The alignment function 140 can also perform alignment by performing three-dimensional mapping based on, for example, five images acquired by the five-image method. More specifically, the alignment function 140 can acquire information on a component in the height direction from the images acquired by the five-image method, and perform alignment using a height component in the CT image. This alignment can improve accuracy of mapping in a back teeth region which is difficult to be obtained in a photograph in an oral cavity.

For example, the alignment function 140 can convert the CT image acquired by the five-image method into a two-dimensional image, and align the two-dimensional image with a photographed image. The alignment function 140 can further perform the three-dimensional mapping by reflecting the two-dimensional image in the CT image.

The alignment function 140 can also perform alignment, for example, by estimating the shape of the teeth exposed from the gums based on the shape of the teeth in the CT image. For example, the alignment function 140 can perform alignment of images by performing alignment processing similar to the above-described processing on the estimated shape of the teeth.

For example, the alignment function 140 can extract a feature portion in the CT image as a landmark and use the landmark for alignment. For example, even in a case where a raised portion or the like in a portion hidden by the gums cannot be acquired as a direct image due to the gums, the alignment function 140 can perform alignment by extracting the raised portion. Further, the alignment function 140 can also extract a landmark from the teeth estimated from the CT image.

The teeth treated in the past, for example, the teeth in which an inlay is embedded or the teeth treated with a crown have features in the images photographed by various photographing techniques. The alignment function 140 can map images using these features as landmarks to perform alignment. For example, a treated portion may have a feature shadow in an X-ray photograph or the like, but alignment can be performed using such a feature.

When treatment similar to the above-described treatment is performed, a feature may be taken as the shape of the teeth instead of a feature in those images. The alignment function 140 can also align teeth that have undergone treatments and the like based on the shape after these treatments. In a case where alignment is performed using the images after the treatment is performed, adjustment can be performed using such features of the shape of the treatment trace.

Furthermore, conversely, in a case where it is possible to estimate that a portion is a trace of treatment, the alignment function 140 can perform alignment by omitting the portion, that is, using a shape or the like other than the features related to the treatment or the like. A timing at which the image is taken is freely selected, and at such any timing, the external shape of the teeth may change when treatment is applied to the teeth. Since the alignment function 140 does not use a change in shape due to treatment as a landmark, it is possible to perform alignment with high accuracy while avoiding these changes. The alignment function 140 may present the alignment together with a case of considering a change in the shape, and a user may select a desired alignment to be used.

Next, the alignment function 140 rotates and adjusts the VR image and the color image in accordance with a direction in which the composite image is to be acquired (S126). For example, the user can designate a desired angle using the VR model. Based on this desired angle, the alignment function 140 acquires the VR image from the VR model.

Furthermore, the alignment function 140 can generate an image acquired from the same angle by using the front image or one of the images in the left and right directions as the color image. In this generation, the alignment function 140 can acquire an angle-shifted image by adjusting the front image to the angle and the position adjusted in S122 and S124, and then combining the front image and one of the images in the left and right directions with any method.

Referring back to FIG. 5, next, the overlay function 142 superimposes the VR image and at least a partial region of the color image (S14).

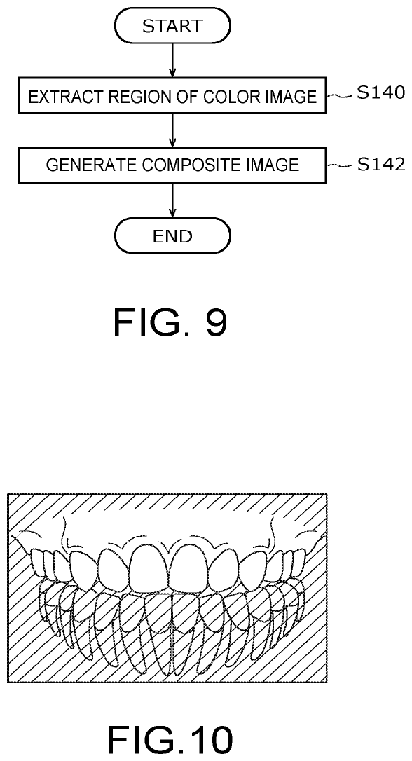

FIG. 9 is a flowchart illustrating processing of the overlay function 142 according to the embodiment. The overlay function 142 first extracts a region to be superimposed from the color image adjusted by the alignment function 140 (S140).

The overlay function 142 superimposes at least an extracted partial region of the color image on the VR image, thereby generating a composite image (S142).

In a case where the alignment function 140 sets the reference points to the same coordinates in position adjustment, the overlay function 142 can combine the images by mapping and superimposing the regions of the color image to be superimposed onto the same coordinates of the VR image.

Furthermore, in a case where the alignment function 140 calculates deviation of the reference point in the position adjustment, the overlay function 142 can combine the images by mapping the region of the color image such that the reference point of the color image and the reference point of the VR image overlap each other.

The overlay function 142 does not necessarily need to perform mapping, and the mapping function may be turned on or off in response to a request from a user.

FIG. 10 is a diagram illustrating an example of the composite image according to the embodiment. As illustrated in this drawing, for example, the medical information processing device 1 can combine the regions of the upper teeth and gums as color images with respect to the VR image.

FIG. 10 is illustrated as a non-limiting example, and the angle and the like of the composite image can be freely and selectively set, for example, in the range where the color image is photographed. In addition, it is possible to similarly generate a superimposed composite image for a state in which the mouth is opened or the like. In addition, for example, it is also possible to combine only teeth not including gums or only gums not including teeth. A boundary between teeth and gums can also be extracted by referring to, for example, a color change in the color image or a shadow in the VR image.

The overlay function 142 may detect a boundary of a lip retractor and exclude a region of the lip retractor from the color image, or may detect a boundary of a mirror and exclude the mirror, as necessary.

Referring back to FIG. 5, the processing circuit 14 executes the processing in S16 to S20 as necessary.

When the transparency is set as a condition, the transparency setting function 144 changes the transparency of the superimposed color image (S16). By changing the transparency, it is possible to simultaneously output a color of the surface of the teeth and gums indicated by the color image and dental caries, treatment traces, and the like indicated by the VR image in a region where the color image is combined.

For example, by appropriately controlling the transparency, it is also possible to perform inspection of periodontal disease while comparing the state of bone absorption in the VR image with the state of the color of the surface in the color image.

When a hue is set as a condition, hue setting function 146 changes the hue of the superimposed color image (S18). By changing the hue, for example, the state of the teeth after the treatment can be output from an aesthetic aspect. As a result, it is easy to receive a request from a patient regarding treatment such as what hue of a prosthesis such as resin or ceramic is emphasized as treatment by the patient.

Further, the hue setting function 146 can also generate a color image in which the position of a light source, brightness of the light source, and the like are set. By combining the generated color images in this manner, the patient and the user can also check a post-treatment state and the like in more various situations.

In a case where the color of the teeth in the color image cannot be accurately acquired in the superimposed image, or in a case where there is missing of the teeth due to metal artifact in the VR image, or the like, the color interpolation function 148 can interpolate the color of the color image or the VR image (S20).

The color interpolation function 148 can interpolate the color of the teeth by any method using a color of surrounding teeth or the like with respect to the surface or the like of teeth for which color information has not been successfully acquired in the color image. Furthermore, also in a case where there is a missing teeth image in the VR image, interpolation can be similarly performed from the teeth image in the surrounding VR image.

The processing circuit 14 may present the composite image to the user via the input/output I/F 10. The user may also provide a further instruction to the presented composite image. For example, in a case where it is necessary to further adjust the position, the angle, the superimposition, the transparency, the hue, or the interpolation, the processing can be continued (S24: NO).

When the processing is continued, the necessary processing from S12 to S20 is performed by changing the condition in S10, and then the edited composite image can be output (S22). In a case where an appropriate composite image has been acquired and the processing has been completed in response to a request from the user (S24: YES), the processing circuit 14 outputs the appropriately composite image, for example, stores the appropriately composite image in the storage unit 12, and ends the processing.

As described above, with the medical information processing device 1 of the present disclosure, it is possible to generate the composite image of the VR image and the color image using a parameter desired by the user. This composite image can assist appropriate diagnosis, treatment, and the like by generating an image in which a state that cannot be visually checked and a state that can be visually checked are appropriately mixed in response to a request from the user.

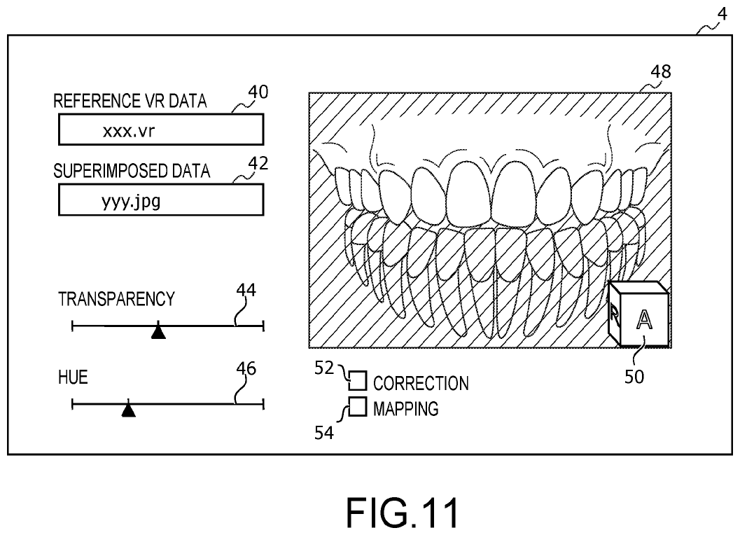

FIG. 11 is a diagram illustrating a non-limiting example of a graphical user interface (GUI) for setting a condition according to the embodiment. As an example, the GUI 4 includes a VR file name input field 40, a superimposed data input field 42, a transparency setting bar 44, a hue setting bar 46, a composite image display region 48, an angle control box 50, a correction selection button 52, and a mapping selection button 54.

The user inputs a data file name indicating a VR model as a reference to the VR file name input field 40 and a data file name indicating a color image to be superimposed in the superimposed data input field 42.

The transparency setting bar 44 includes a slide bar, and can set the transparency of the color image by sliding on the slide bar.

Similarly, the hue setting bar 46 can set the hue by sliding on the bar.

The composite image is displayed in the composite image display region 48. The user may be able to designate the region of the color image by selecting a portion to be superimposed in the composite image. This designation may be executed by dividing the region into upper teeth, lower teeth, upper gums, lower gums, and the like, and combining the color image at a selected location. As another example, the user may designate a combined region by surrounding a region with a lasso tool. In addition, a button for separately selecting a region, for example, a button for designating a region such as upper teeth or lower teeth may be prepared, and a region to be overlapped with the color image may be designated by selecting this button.

The angle control box 50 may be installed in the composite image display region 48. The angle control box 50 may be moved by, for example, a mouse drag operation or the like to change the angle of the VR image, and accordingly, the color image processed by the processing circuit 14 maybe combined and displayed.

Furthermore, the correction selection button 52 and the mapping selection button 54 may be provided. These buttons may be used to allow the user to select whether to perform each of correction and mapping.

In the above description, an example using the CT image has been described, but the present invention is not limited thereto, and for example, an MRI image can also be used.

In the above embodiment, the input interface can be implemented by a trackball for performing various settings, a switch button, a mouse, a keyboard, a touch pad for performing an input operation by touching an operation surface, a touch screen in which a display screen and the touch pad are integrated, a non-contact input circuit using an optical sensor, a sound input circuit, and the like. The input interface is connected to a control circuit, converts an input operation received from an operator into an electrical signal, and outputs the electrical signal to the control circuit. It is noted that, in the present specification, the input interface is not limited to one including physical operation components such as a mouse and a keyboard. For example, examples of the input interface include an electrical signal processing circuit that receives an electrical signal corresponding to an input operation from an external input device provided separately from the device and outputs the electrical signal to the control circuit.

Additionally, in the above embodiment, each processing function of the information processing functions is recorded in a storage circuit in the form of a program executable by a computer. The processing circuit can include a processor. For example, the processing circuit reads a program from the storage circuit and executes the program to implement a function corresponding to each program. In other words, the processing circuit in a state of reading each program has each function illustrated in the processing circuit illustrated in the drawings. It is noted that, in the drawings, it has been described that each processing function is implemented by a single processor. However, the processing circuit may be configured by combining a plurality of independent processors, and the functions thereof may be implemented by each processor executing the program. Furthermore, in the drawings, it has been described that a single storage circuit stores a program corresponding to each processing function, but a plurality of storage circuits may be arranged in a distributed manner, and the processing circuit may read the corresponding program from each storage circuit.

In the above description, an example has been described in which the “processor” reads a program corresponding to each function from the storage circuit and executes the program, but the embodiment is not limited thereto. The term “processor” can mean, for example, a circuit such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). In a case where the processor is, for example, the CPU, the processor implements a function by reading and executing a program stored in the storage circuit. On the other hand, when the processor is the ASIC, instead of storing the program in the storage circuit, the function is directly incorporated as a logic circuit in a circuit of the processor. It is noted that each processor of the present embodiment is not limited to being configured as a single circuit for each processor, and a plurality of independent circuits may be combined to be configured as one processor to implement the function. Further, a plurality of components in the drawings may be integrated into one processor to implement the function.

According to at least one embodiment described above, it is possible to execute composition of a VR image and a color image based on a user's desired condition.

Although several embodiments have been described above, these embodiments have been presented only as examples, and are not intended to limit the scope of the invention. These embodiments can be implemented in various other forms, and various omissions, substitutions, changes, and combinations of the embodiments can be made without departing from the gist of the invention. These embodiments and modifications thereof are included in the scope and gist of the invention and are included in the invention described in the claims and the scope equivalent thereto.

1. A medical information processing device comprising:
a memory; and
a processor configured to:
generate a volume rendering (VR) image from a medical image of a subject;
acquire a color image acquired by one or a plurality of optical imaging units, the color image sharing at least a part of an imaging region with the medical image; and
superimpose at least a part of the color image on the VR image.

2. The medical information processing device according to claim 1, wherein
the processor is configured to extract a common feature from each of the VR image and the color image, and to align the VR image and the color image based on the feature.

3. The medical information processing device according to claim 2, wherein
the medical image is a computed tomography (CT) image in an oral cavity, and
the processor is configured to align the VR image and the color image based on a line segment connecting end points of teeth and a surface area of the teeth.

4. The medical information processing device according to claim 2, wherein
the medical image is a CT image in an oral cavity, and
the processor is configured to perform alignment in consideration of a height component of the CT image and an image on which three-dimensional mapping has been executed.

5. The medical information processing device according to claim 2, wherein
the medical image is a CT image in an oral cavity, and
the processor is configured to reconstruct, in three dimensions, an image obtained by performing alignment using an image obtained by converting the CT image into two dimensions.

6. The medical information processing device according to claim 2, wherein
the processor is configured to estimate shapes of teeth exposed from gums and to perform alignment.

7. The medical information processing device according to claim 2, wherein
the processor is configured to perform alignment by using shapes of treated teeth or features in the treated teeth.

8. The medical information processing device according to claim 2, wherein
the processor is configured to perform alignment without using a feature of a change in shapes of teeth, the shapes of the teeth having been changed by treatment.

9. The medical information processing device according to claim 1, wherein
the processor is configured to set transparency of the color image to be superimposed on the VR image.

10. The medical information processing device according to claim 1, wherein
the processor is configured to set a hue of the color image to be superimposed on the VR image.

11. The medical information processing device according to claim 1, wherein
the processor is configured to interpolate, when color information in at least a partial region of the color image to be overlaid on the VR image is not acquirable, the color information on the region from surrounding color information.

12. The medical information processing device according to claim 1, wherein
the color image is an image in an oral cavity, the image being acquired by a five-image method.

13. A medical information processing method comprising:
generating, by a processor, a VR image from a medical image of a subject;
acquiring, by the processor, a color image acquired by one or a plurality of optical imaging units, the color image sharing at least a part of an imaging region with the medical image; and
superimposing, by the processor, at least a part of the color image on the VR image.

14. A non-transitory computer readable medium having a program stored therein and configured to cause a processor to execute:
generating a VR image from a medical image of a subject;
acquiring a color image acquired by one or a plurality of optical imaging units, the color image sharing at least a part of an imaging region with the medical image; and
superimposing at least a part of the color image on the VR image.
